(19) United States
(12) Patent Application Publication
(10) Pub. No.: US 2023/0258021 A1
(43) Pub. Date: Aug. 17, 2023

(54) APPARATUS FOR EFFICIENTLY TRANSFERRING ELECTRIC POWER TO DOORS IN ORDER TO ENERGIZE DOOR-ASSOCIATED ELECTRICAL DEVICES

(71) Applicant: Tom Chi, Wichita, KS (US)

(72) Inventor: Tom Chi, Wichita, KS (US)

(21) Appl. No.: 18/010,573

(22) PCT Filed: Jun. 18, 2021
(86) PCT No.: PCT/US2021/038003

§ 371 Date: Dec. 15, 2022

Related U.S. Application Data
(60) Provisional application No. 63/040,663, filed on Jun. 18, 2020.

(51) Int. Cl. E05B 15/02; E05B 1/00; H02G 3/00; A61L 2/26; A61L 2/10

(52) U.S. Cl. CPC E05B 15/0205; E05B 1/0069; H02G 3/00; A61L 2/26; A61L 2/10; A61L 2202/11

(57) ABSTRACT

An apparatus for efficiently transferring electric power to doors from a door jamb in order to energize door-associated electrical devices, which includes a door for entering and exiting a room, a stationary door electric power transfer bar affixed to the door and a door jamb associated with the door. A striker plate is provided for the door jamb and there is a retractable energized electric power transfer member in the striker plate, along with a power supply for electrical devices associated with the door which would require electrical power.

### BACKGROUND OF INVENTION

### Technical Field of the Invention

The present invention relates generally to an apparatus for efficiently transferring electric power to doors in order to energize door associated electrical devices, such as doorknobs, door levers, door bar handles, door plates and other similar devices in order to connect the devices with electrical energy to energize for example, sanitizing ultraviolet C sterilizing germicidal light enabled door knobs, door levers, door bar handles and door plates and other devices that come into contact with hands so that the surfaces are initially germ-free and remain germ-free.

### Description of the Prior Art

It is well established that germs from hands are easily communicated to a person's face especially when the hands touch the face several times a minute, as for example the eyes are rubbed, the nose is touched and other times when the hands consciously and unconsciously touch the face. Since the exposed face, more than any other part of the human body, is more susceptible to transmitting germs to the body's systems it imperative to maintain as best as possible a germ free face and hands free of pathogens which can enter the eyes, ears, mouth, nose, etc.

The prior art known to the inventor is represented by the following: Littles, U.S. Pat. No. 4,777,570, issued Oct. 11, 1988; Geringer et al., U.S. Patent Application Publication No. 2016/0273243, published Sep. 22, 2016; Martin et al., U.S. Patent Application Publication No. 2018/0080253, published Mar. 22, 2018; Martin et al., U.S. Patent Application Publication No. 2018/0171669, published Jun. 21, 2018; and, Gerhardt et al., U.S. Patent Application Publication No. 2019/0342443.

### SUMMARY OF THE INVENTION

An object of the present invention for efficiently transferring electric power to doors in order to energize door-associated electrical devices is to provide an apparatus to safely and efficiently supply electric power to door devices in order to connect with and energize sanitizing ultraviolet C sterilizing germicidal light enabled doorknobs, door levers, door bar handles, door plates, door strike plates and other door accessories.

Another object of the present invention is to provide for the transmission of electric power to door mechanisms including to recharge batteries, lights, emergency exit signs, video displays, fans and many other purposes with the primary purpose of the invention to efficiently transfer power to doorknobs, door levers, door bar handles, door plates, door strike plates and other door accessories for any number of uses including UV sterilization of doorknobs, door levers, door bar handles, door plates, door strike plates and other door accessories.

A still further object of the invention to provide electric power for door enabled thru-door fans, smoke alarms and other electrical devices.

Another object of the present invention is to provide a low cost, user friendly, easy to install wiring system to transmit electrical power to door accessories.

Yet another object of the invention is to utilize the transferred electric power for any purpose such as for observational cameras, for night and other lighting and may include energizing ultraviolet lights for sterilization of doorknobs, pull handles, door levers, door push plates etc, for the purpose of maintaining sterility and decontaminating contaminated surfaces from germs.

A still further object of the invention permits the conveyed power for use with automatic locks and/or other devices.

A further object of the present invention is to incorporate into a doorknob, door lever and other devices sanitizing ultraviolet C sterilizing germicidal light UV light so that any germs on the users hands are dissipated.

The foregoing and related objects are accomplished by an apparatus for efficiently transferring electric power to doors from a door jamb in order to energize door-associated electrical devices, which includes a door for entering and exiting a room, a stationary door electric power transfer bar affixed to said door and a door jamb associated with the door. A striker plate is provided for the door jamb and there is a retractable energized electric power transfer member in the striker plate, along with a power supply for electrical devices associated with the door which would require electrical power.

Preferably, the means for delivering electric power to the door-associated electrical devices is a stationary door electrical power transfer bar affixed to the door which communicates with a retractable energized electric power transfer member in the striker plate.

It is further preferable that the door associated-electrical device is a stationary door electrical power transfer bar affixed to the door having stationary electrical contacts so that when the door is closed said contacts come into contact with contacts on the retractable energized electric power transfer member in the striker plate creating an energized electrical circuit.

Examples of associated electrical devices requiring associated with the door may include one or more doorknobs enabled with sanitizing ultraviolet C sterilizing germicidal light, door levers enabled with sanitizing ultraviolet C sterilizing germicidal light, door bar handles doorknobs enabled with sanitizing ultraviolet C sterilizing germicidal light, door plates doorknobs enabled with sanitizing ultraviolet C sterilizing germicidal light, door strike plates doorknobs enabled with sanitizing ultraviolet C sterilizing germicidal light, recharge batteries, lights, emergency exit signs, video devices and fans.

Ultraviolet light enabled doorknobs and door handles maintain a germ-free surface by disinfecting and sterilizing doorknobs, door handle levers, door bar handles, door plates and other devices that are touched by a user hands. By exposing the doorknob, door lever, door bar handle, door plates, faucet lever/handle, elevator buttons and other devices to ultraviolet sterilizing light to kill or inactivate microorganisms by destroying nucleic acids, disrupting their DNA/RNA and leaving them unable to function or reproduce.

Other objects and features of the present invention will become apparent when considered in combination with the accompanying drawing figures, which illustrate certain preferred embodiment of the present invention. It should, however, be noted that the accompanying drawing figures are intended to illustrate only select preferred embodiments of the claimed invention and are not intended as a means for defining the limits and scope of the invention.

### BRIEF DESCRIPTION OF THE DRAWING FIGURES

In the drawing figures, wherein similar features are denoted with similar reference numerals throughout the several views:

FIG. 1 is front elevational view of the apparatus for efficiently transferring electric power to doors in order to energize door associated electrical devices and specifically shown is a sanitizing ultraviolet C sterilizing germicidal light enabled doorknob;

FIG. 2 is a front perspective elevational view detailing the stationary or associated power transfer bar and the mating retractable power transfer strike plate in the door frame/jamb;

FIG. 3 is a cross-sectional view of the stationary door-associated power transfer bar and the mating retractable power transfer member; and,

FIG. 4 is a more detailed a cross-sectional view of the stationary door-associated power transfer bar and the mating retractable power transfer member.

### DESCRIPTION OF THE REFERENCE NUMERALS

10 door

12 wall

14 hinge

16 door frame/jamb

18 doorknobs, door levers, door bar handles, door push plates, door strike plates all enabled with sanitizing ultraviolet C sterilizing genocidal light

20 standard door latch in door

20a standard striker plate in door jamb

22 stationary door electric power transfer bar affixed to door

22a stationary electric power contacts on stationary electric power transfer bar that is affixed to door

24 retractable energized electric power transfer member in striker plate door jamb

24a electric power contacts on retractable energized electric power transfer member in striker plate door jamb

24b striker plate in door jamb

28 electric power source

### DETAILED DESCRIPTION OF THE DRAWING FIGURES AND PREFERRED EMBODIMENTS

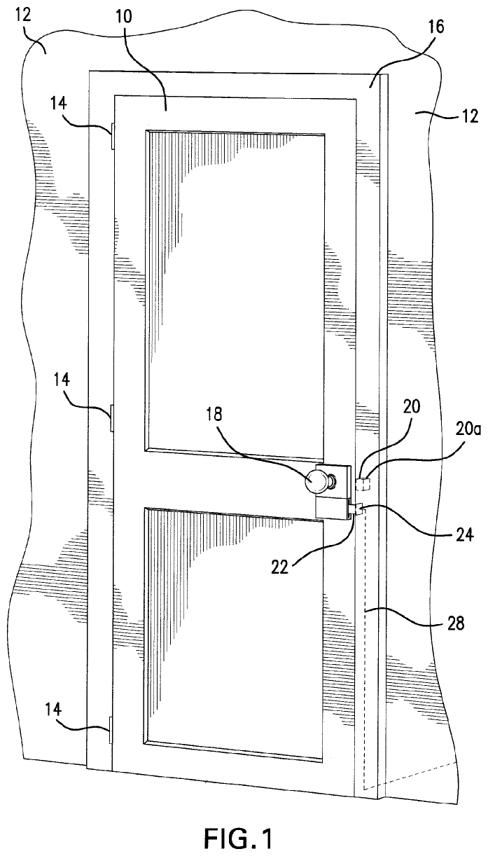

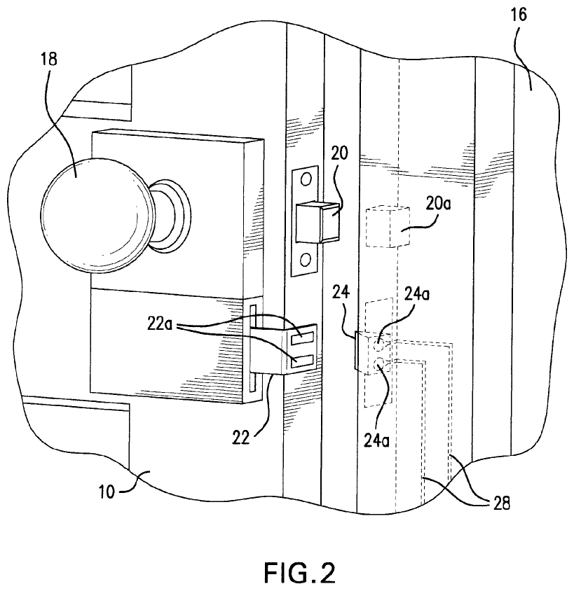

Turning now, in detail, to the accompanying drawing figures, FIG. 1 and FIG. 2 shows the structure of the apparatus for efficiently transferring electric power to doors in order to energize door associated electrical devices and specifically shown is sanitizing ultraviolet C sterilizing germicidal light enabled doorknob 18. Other electrical door associated devices may also be powered and include rechargeable batteries, lights, emergency exit signs, video devices and fans. Door 10 is attached to wall 12 by a plurality of hinges 14 and opens and closes as a typical door. Door 10 is associated with door frame and jamb 16 in wall 10. Standard door latch 20 in door 10 engages with standard striker plate 20a in door/frame 16. Sanitizing ultraviolet C sterilizing germicidal light enabled doorknob 18 is electrically powered by receiving electrical power from stationary door electric power transfer bar 22 which in attached to door 10.

Affixed to door frame/jamb 16 is retractable energized electric power transfer member 24. When door 10 is closed electric power contacts 24a and 24b on retractable energized electric power transfer member 24 come into contact with stationary electric power contacts 22a on stationary electric power transfer bar 22 and energize an electric circuit to power door associated electric devices. Electric power 28 is communicated to retractable energized electric power transfer member contacts 24a contained on retractable energized electric power transfer member 24 within striker plate 24b.

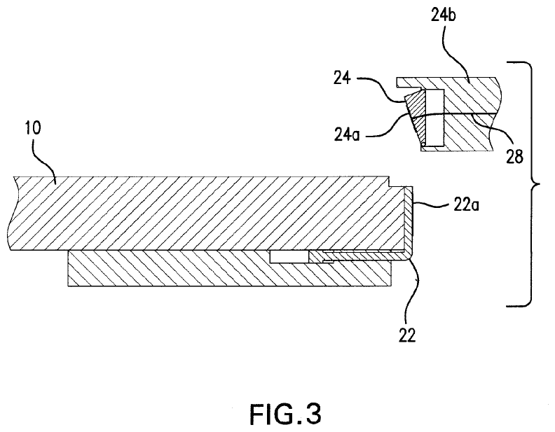

FIG. 3 shows, in an off-set manner, stationary door electric power transfer bar 22 from retractable energized electric power transfer member 24 in striker plate 24b. When door 10 is closed stationary electric power contacts 22a on stationary door electric power transfer bar 22 engage with electric power contacts 24a on retractable energized electric power transfer member 24, thus completing an electrical circuit to power door related accessories. As shown retractable energized electric power transfer member in striker plate 24b is not engaged with stationary door electric power transfer bar 22 resulting in an open electrical circuit.

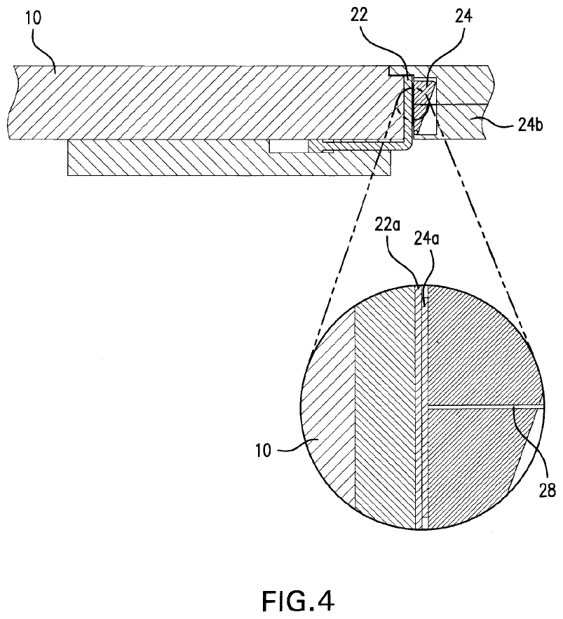

FIG. 4 shows communication of stationary door electric power transfer bar 22 with retracted retractable energized electric power transfer member 24 in striker plate 24b resulting in an energized electric circuit that transmit electric power to door 10 and to the door associated devices such as doorknob enabled sanitizing ultraviolet C germicidal light 18.

Stationary electric power contacts 22a on stationary door electric power transfer bar 22 affixed to door 10 is shown in communication with electric power contacts 24a on retractable energized electric power transfer member 24 in striker plate 24b in door/frame jamb 16.

While only several embodiments of the present invention have been shown and described, it will be obvious to those skilled in the art that many modifications may be made to the present invention without departing from the spirit and scope thereof.

What is claimed is:

1. An apparatus for transferring electric power to a door from a door jamb for energizing door-associated an electrical device, comprising:
a door for entering and exiting a room;
a stationary door electric power transfer bar affixed to said door;
a door jamb for the door;
a striker plate for the door jamb;
a retractable energized electric power transfer member in said striker plate; and,
means for delivering electric power an electrical device associated with said door.

2. The apparatus for transferring electric power to a door from a door jamb for energizing door-associated an electrical device according to claim 1, wherein said means for delivering electric power to the electrical device associated with the door a stationary door electrical power transfer bar affixed to door with a retractable energized electric power transfer member in said striker plate.

3. The apparatus for transferring electric power to a door from a door jamb for energizing door-associated an electrical device according to claim 1, further comprising a stationary door electrical power transfer bar affixed to said door having stationary electrical contacts so that when said door is closed said stationary electrical contacts come into contact with contacts on said retractable energized electric power transfer member in said striker plate creating an energized electrical circuit.
